(19) United States
(12) Patent Application Publication
(10) Pub. No.: US 2026/0041408 A1
(43) Pub. Date: Feb. 12, 2026

(54) NASAL SPECIMEN COLLECTION SYSTEM

(71) Applicant: Board of Regents of the University of Nebraska, Lincoln, NE (US)

(72) Inventors: Thang Nguyen, Elkhorn, NE (US); Michael Wadman, Omaha, NE (US); Wesley Zeger, Omaha, NE (US)

(21) Appl. No.: 19/099,310

(22) PCT Filed: Aug. 1, 2023
(86) PCT No.: PCT/US2023/071416

§ 371 Date: Jan. 28, 2025

Related U.S. Application Data
(60) Provisional application No. 63/395,606, filed on Aug. 5, 2022.

(51) Int. Cl. A61B 10/00; A61M 3/02

(52) U.S. Cl. CPC A61B 10/00; A61M 3/0279

(57) ABSTRACT

A nasal specimen collection apparatus comprises: a nasal pathway irrigator including an irrigation chamber that defines an irrigation path for directing fluid into a nasal pathway to dislodge a sample from within the nasal pathway; a nasal sample collector including a collection chamber that surrounds the nasal pathway irrigator, wherein the collection chamber has an outer rim that is configured to interface with the nasal pathway, and wherein the collection chamber defines a collection path for recapturing at least a portion of the fluid to collect the sample that was dislodged from within the nasal pathway; and an annular shell that surrounds the nasal sample collector with an annular cavity formed in between the annular shell and the collection chamber, wherein the annular shell is configured to interface with a sample tube so that contents of the collection chamber can be emptied into the sample tube.

### CROSS-REFERENCE TO RELATED APPLICATIONS

The present application claims priority under 35 U.S. C. § 119(e) to U.S. Provisional Application Ser. No. 63/395,606, filed Aug. 5, 2022, and titled “NASAL SPECIMEN COLLECTION SYSTEM,” which is incorporated herein by reference in its entirety.

### TECHNICAL FIELD

The present invention relates to apparatus, systems, and methods for collection of nasal specimens.

### BACKGROUND

Widespread diagnostic testing for the SARS-COV-2 virus was a limiting factor in efforts to accurately project case numbers and contain the disease known as COVID-19. After the first confirmed case of COVID-19 in China, the virus quickly became the center of a global pandemic resulting in economic and social shutdowns. Global supply and personnel shortages stifled the deployment of testing options for patients, hindering downstream processes such as contact tracing and containment. This problem was further compounded by barriers that prevented patients from accessing testing resources.

With the resurgence of endemic respiratory tract infections (RTI) such as influenza, it is paramount to have reliable alternative testing modalities to bolster the current infrastructure. This has led to an influx of academic and corporate interest in developing alternative testing methods for RTI.

The nasopharyngeal swab is the gold standard collection method for COVID-19 and other RTI, due to data suggesting a higher viral concentration in the nasopharyngeal cavity. However, literature suggests lower patient acceptance of the nasopharyngeal swab collection method with procedural discomfort attributing to the low acceptance rate. This procedure is somewhat invasive and traumatizing for patients as it requires deep probing of the posterior nasopharynx with a stiff swab applicator. The nasopharyngeal swab procedure has been known to cause pain and injuries such as epistaxis. Additionally, there is also a considerable infection risk to healthcare workers administering the nasopharyngeal swab as patients tend to cough or sneeze during the procedure.

Given the state of the art, there is a need for alternative collection methods that are more comfortable but can produce adequate specimens when compared to a nasopharyngeal swab. There is also need for diagnostic tests that can be self-administered to prevent/reduce the risk of contagion.

### SUMMARY

A nasal specimen collection (NSC) apparatus is disclosed. In embodiments, the NSC apparatus includes a nasal pathway irrigator nested within a nasal sample collector. The nasal pathway irrigator includes an irrigation chamber that defines an irrigation path for directing fluid into a nasal pathway in order to dislodge a sample from within the nasal pathway. The nasal sample collector includes a collection chamber that surrounds the nasal pathway irrigator, wherein the collection chamber has an outer rim that is configured to interface with the nasal pathway. The collection chamber also defines a collection path for recapturing at least a portion of the fluid that was directed into the nasal pathway by the irrigator in order to collect the sample that was dislodged from within the nasal pathway. The NSC apparatus further includes an annular shell forming a cap-like structure that surrounds the nasal sample collector with an annular cavity formed in between the annular shell and the collection chamber of the nasal sample collector. The annular shell is configured to interface with a sample tube so that contents of the collection chamber can be emptied into the sample tube.

The NSC apparatus may be implemented within a NSC system (e.g., a diagnostic kit) that further includes a fluid actuator and a sample tube that are configured to interface with the NSC apparatus. In embodiments, the nasal pathway irrigator and the nasal sample collector are nested within the annular shell of the NSC apparatus and supported by an annular base. The annular base includes an irrigation port for coupling the fluid actuator to the irrigation chamber of the nasal pathway irrigator. When the fluid actuator (e.g., a syringe) is coupled to the irrigation chamber via the irrigation port, the fluid actuator can be used to direct fluid along the irrigation path running through the irrigation chamber to deliver the fluid into the nasal pathway in order to dislodge a sample from within the nasal pathway. The nasal sample collector then receives the sample by recapturing at least a portion of the fluid (now containing the sample) within the collection chamber via the interface between the outer rim of the collection chamber and the nasal pathway. The fluid is preferably directed into the nasal pathway at an incline so that at least a portion of the fluid will flow back down into the collection chamber. After collecting a sample, the annular shell may be interfaced with (e.g., mated with or coupled to) the sample tube so that contents of the collection chamber, including recaptured fluid and the collected sample, can be emptied into the sample tube.

A method of nasal specimen collection may employ the NSC apparatus or a similarly structured device. The method may include the steps of: (1) providing a nasal specimen collection apparatus that includes a nasal pathway irrigator and a nasal sample collector nested within an annular shell that has a coupling interface (e.g., cooperative threading); (2) inserting a distal end of the nasal sample collector into a nasal pathway, wherein the nasal sample collector includes a collection chamber that surrounds the nasal pathway irrigator and has an outer rim that is configured to interface with the nasal pathway; (3) directing fluid into the nasal pathway via the nasal pathway irrigator in order to dislodge a sample from within the nasal pathway; (4) recapturing at least a portion of the fluid within the collection chamber of the nasal sample collector in order to collect the sample that was dislodged from within the nasal pathway; and (5) coupling the annular shell to a sample tube via the coupling interface to transfer contents of the collection chamber to the sample tube and to seal the contents within the sample tube.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The Detailed Description is provided with reference to the accompanying Drawings. The use of the same reference numbers in different instances in the Detailed Description and the Drawings may indicate similar or identical items. The Drawings are not necessarily to scale, and any disclosed processes may be performed in an arbitrary order, unless a certain order of steps/operations is inherent or specified in the Detailed Description or in the Claims.

FIG. 1A is a perspective view of a nasal specimen collection (NSC) apparatus, in accordance with one or more embodiments of this disclosure.

FIG. 1B is a side elevation view of the NSC apparatus, in accordance with one or more embodiments of this disclosure.

FIG. 1C is a cross-sectional view of the NSC apparatus, in accordance with one or more embodiments of this disclosure.

FIG. 2A is a perspective view of the NSC apparatus being coupled to a fluid actuator configured to drive fluid through a nasal pathway irrigator of the NSC apparatus to dislodge a sample from within a nasal pathway, wherein at least a portion of the fluid is recaptured by a nasal sample collector that surrounds the nasal pathway irrigator, in accordance with one or more embodiments of this disclosure.

FIG. 2B is a side elevation view of the NSC apparatus being coupled to the fluid actuator, in accordance with one or more embodiments of this disclosure.

FIG. 2C is a cross-sectional view of the NSC apparatus coupled to the fluid actuator, in accordance with one or more embodiments of this disclosure.

FIG. 3A is a perspective view of the NSC apparatus being coupled to a sample tube configured to receive the recaptured fluid and dislodged sample from within the nasal sample collector of the NSC apparatus, in accordance with one or more embodiments of this disclosure.

FIG. 3B is a side elevation view of the NSC apparatus being coupled to the sample tube, in accordance with one or more embodiments of this disclosure.

FIG. 3C is a cross-sectional view of the NSC apparatus coupled to the sample tube, in accordance with one or more embodiments of this disclosure.

FIG. 3D is a cross-sectional view of the NSC apparatus coupled to the sample tube, in accordance with one or more embodiments of this disclosure.

FIG. 4A is a perspective view of the NSC apparatus being coupled to a tip adapter that can come in different sizes to accommodate pediatric patients or adults with small nostrils, in accordance with one or more embodiments of this disclosure.

FIG. 4B is a side elevation view of the NSC apparatus being coupled to the tip adapter, in accordance with one or more embodiments of this disclosure.

FIG. 4C is a perspective view of the NSC apparatus coupled to the tip adapter, in accordance with one or more embodiments of this disclosure.

FIG. 4D is a side elevation view of the NSC apparatus coupled to the tip adapter, in accordance with one or more embodiments of this disclosure.

FIG. 4E is a cross-sectional view of the NSC apparatus coupled to the tip adapter, in accordance with one or more embodiments of this disclosure.

FIG. 5 is a cross-sectional view of a NSC apparatus coupled to a sample tube, in accordance with one or more embodiments of this disclosure.

### DETAILED DESCRIPTION

The present disclosure is directed to a nasal specimen collection (NSC) apparatus with preferably concentric irrigation and collection paths. However, alternative structural configurations are also contemplated (e.g., side-by-side, overlapping/integrated, or off-axis fluid paths). The irrigation path is configured to direct fluid into a nasal pathway in order to dislodge a sample (sometimes referred to herein as a “specimen”) from the nasal pathway, and when the fluid flows back out of the nasal pathway, the collection path is configured to direct at least a portion of the fluid and the sample dislodged from the nasal pathway into a collection chamber. As used herein, the term “nasal pathway” includes any anatomical space extending from a human/animal subject's nostril as far as (and possibly including) their oral pharynx.

In exploring a potential alternative nasopharyngeal specimen collection method, the inventors developed the concept of nasopharyngeal debridement using fluid irrigation. This alternative respiratory pathogen collection device (the NSC apparatus) was designed to be self-administered by the patient but can also be administered by a healthcare professional. The NSC apparatus irrigates the patient's nasopharyngeal cavity with a fluid (e.g., saline solution), debriding epithelial cells potentially harboring respiratory pathogens. The irrigation solution is immediately recaptured into a self-contained collection chamber within the NSC apparatus, minimizing the need to handle infectious bodily fluids. By the nature of a self-administered and self-contained device, there is considerably less infection risk for healthcare professionals. It is also contemplated that replacement of the traditional nasopharyngeal swab with a fluid debridement mechanic will make the procedure less invasive, resulting in higher patient acceptance.

Through the course of developing the NSC apparatus, the inventors determined that sufficient pathological sampling can be achieved by mechanism of nasopharyngeal irrigation that is proportionate to the nasopharyngeal swab method. Using data from wound care literature, it was determined an irrigation pressure of 5 to 15 PSI (pounds per square inch) would be sufficient to overcome the pathogen adhesion threshold. Taking this information into account, embodiments of the NSC apparatus described herein have been designed to apply an irrigation pressure of 5 to 20 PSI but not to exceed 30 PSI. However, the NSC apparatus may function appropriately at other irrigation pressures depending on the overall device structure and requirements of the application.

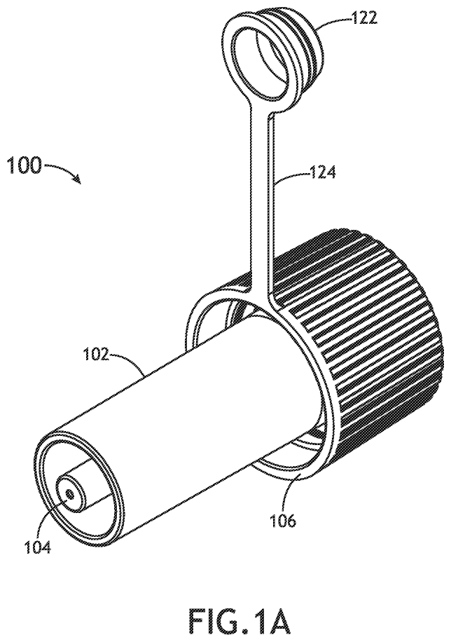

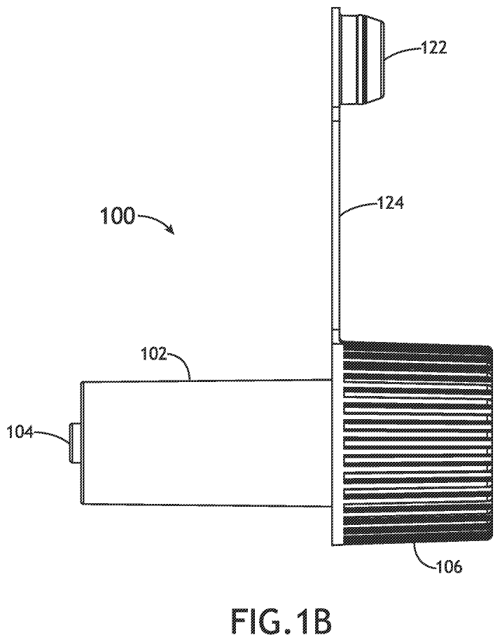

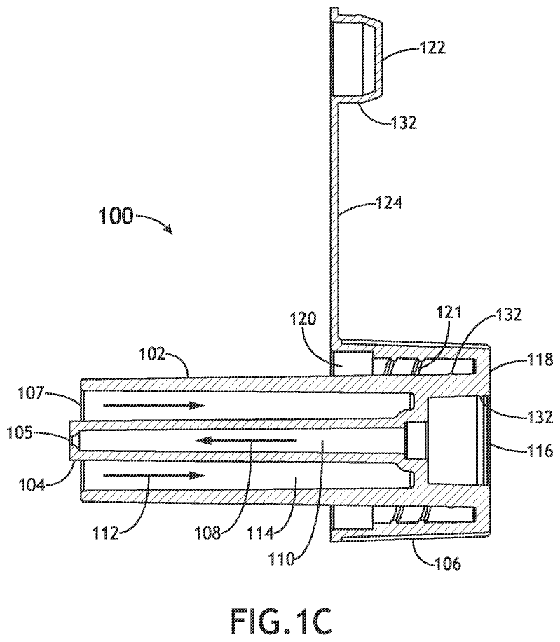

FIGS. 1A through 1C illustrate a NSC apparatus 100, in accordance with one or more embodiments of the present disclosure. The NSC apparatus 100 preferably has concentric inner and outer nozzles at its distal end, where the outer nozzle is configured to interface with a nasal pathway. For example, the outer nozzle may be tapered to fit into a nostril and partially extend into the nasal pathway so that fluid can flow from the inner nozzle into the nasal pathway, and when the fluid flows back out of the nasal pathway, at least a portion of the fluid (preferably all/most of the fluid) can be recaptured via the outer nozzle. However, alternative structural configurations are also contemplated (e.g., side-by-side, overlapping/integrated, or off-axis fluid injection/collection nozzles). In embodiments, the nozzles may be at least partially conical (e.g., truncated cones), dome-like, substantially cylindrical (e.g., true cylinders, tapered cylinders, barrel-shaped), at least partially angled/slanted, or any combination of the foregoing shapes, among others.

The NSC apparatus 100 includes a nasal pathway irrigator 104 nested within a nasal sample collector 102, wherein the distal ends of the nasal pathway irrigator 104 and the nasal sample collector 102 define the inner and outer nozzles (see FIG. 1C, openings 105 and 107), respectively.

As shown in FIG. 1C, the nasal pathway irrigator 104 includes an irrigation chamber 110 that defines an irrigation path 108 for directing fluid into a nasal pathway in order to dislodge a sample from within the nasal pathway. The irrigation chamber 110 may define an open pathway (irrigation path 108) from a proximal end of the NSC apparatus 100 to its distal end. In this regard, the irrigation chamber 110 may be considered an irrigation channel/lumen as it is an open-ended fluid path. The term “chamber” should be understood as including either open or closed ended spaces that define a fluid path or compartment. The irrigation chamber 110 is configured to be coupled to a fluid actuator (e.g., a syringe) via an irrigation port 116.

The nasal sample collector 102 includes a collection chamber 114 that surrounds the nasal pathway irrigator 104. The collection chamber 114 has an outer rim at its distal opening 107 that is configured to interface with the nasal pathway. The collection chamber 114 defines a collection path 112 for recapturing at least a portion of the fluid (preferably all/most of the fluid) that was directed into the nasal pathway by the irrigator 104 in order to collect the sample that was dislodged from within the nasal pathway. In preferred embodiments, the collection chamber 114 has a substantially cylindrical body with the irrigation chamber 110 extending longitudinally through a center of the substantially cylindrical body, such that the collection chamber 114 has an annular cross-section at almost any given point along its longitudinal axis.

As shown in FIG. 1C, the openings 105 and 107 may be circular and annular, respectively. However, in other embodiments, the NSC apparatus 100 may have a different number of openings 105 for the irrigation path 108, a different number of openings 107 for the irrigation path 112, and/or a different arrangement of the openings 105 and 107. In preferred embodiments, the irrigation path 108 and the collection 112 are coaxial. For example, the opening 105 for the irrigation path 108 may be centered and surrounded by the opening 107 for the collection path 112. However, in other embodiments, the irrigation path 108 and the collection path 112 may be arranged differently (e.g., reversed, arranged side-by-side, one only partially surrounding the other, etc.).

The NSC apparatus 100 is configured to inject fluid into the nasal pathway via the distal opening 105 of the nasal pathway irrigator 104 and then recapture at least a portion of the fluid (preferably most or substantially all of the fluid) via the distal opening 107 of the nasal sample collector 102.

The irrigation chamber 110 and the collection chamber 114 are preferably separate and coaxial chambers/fluid pathways as shown in FIG. 1C. However, alternative structural configurations are also contemplated (e.g., side-by-side, overlapping/integrated, or off-axis fluid irrigation/collection chambers/paths). As noted above, the irrigation path 108 is configured to direct a fluid (e.g., saline solution) from the irrigation chamber 110 into the nasal pathway in order to dislodge (e.g., debride or otherwise remove) a sample (e.g., epithelial cells or other specimen) from the nasal pathway. The interior of the irrigation chamber 110 may be tapered (with a progressively reduced diameter) to provide appropriate fluid pressure and flow rate. For example, the interior irrigation chamber 110 may be tapered in the range of 0.5 to 5 degrees from its base to distal end. In a preferred embodiment, the interior irrigation chamber 110 includes at least a first section and a second section, wherein the first (proximal) section is tapered in the range of 0.1 to 5 degrees over a distance in the range of 20 to 50 millimeters and the second (distal) section is tapered in the range of 15 to 45 degrees over a distance in the range of 0.5 to 5 millimeters. The inventors have found that desired flow rates and fluid pressures can be achieved with better manufacturability (e.g., better injection molding outcomes) by having two tapered sections, one section being slightly tapered and the other section being increasingly tapered, as described above. However, the irrigation chamber 110 does not necessarily have to be tapered and may have a constant diameter in other embodiments.

When the fluid flows back out of the nasal pathway, the collection path 112 is configured to direct at least a portion of the fluid and the sample dislodged from the nasal pathway into the collection chamber 114. The collection chamber 114 may be configured to recapture 1 to 10 milliliters of irrigation fluid. For example, in some embodiments, the collection chamber 114 is capable of holding up to 3 milliliters of fluid. In other embodiments, the collection chamber 114 may have a higher/lower capacity depending on application requirements.

To conform to the shape of a typical nostril, the outer body of the collection chamber 114 may also be tapered. The outer body of the collection chamber 114 can also include multiple sections (e.g., a non-tapered or minimally tapered section and an increasingly tapered section), but the inventors have found that a slight, constant taper along the outer body of the collection chamber 114 results in improved manufacturability (e.g., better injection molding outcomes) of the NSC apparatus 100. In preferred embodiments, the outer body of the collection chamber 114 is tapered in the range of 0.5 to 5 degrees from its base to distal end. However, the outer body of the collection chamber 110 does not necessarily have to be tapered and may have a constant diameter in other embodiments.

In embodiments, the nasal pathway irrigator 104 extends past the outer rim of the nasal sample collector 102 to prevent recaptured fluid from backflowing from the collection chamber 114 into the irrigation chamber 110. For example, the distal tip/nozzle defined by opening 105 of the irrigation chamber 110 may extend past the distal tip/nozzle of the collection chamber 114 defined by opening 107. In preferred embodiments, the distal end of the nasal pathway irrigator 104 extends approximately 0.1 to 5 millimeters past the distal end of the nasal sample collector 102. However, other dimensions may be appropriate, and the nasal pathway irrigator 104 does not necessarily have to extend past the distal end of the nasal sample collector 102. In some embodiments, backflow may prevented using other techniques. For example, the nasal pathway irrigator 104 may include a one-way valve.

The NSC apparatus further includes an annular shell 106 that surrounds the nasal sample collector 102 with an annular cavity 120 formed in between the annular shell 106 and the collection chamber 114 of the nasal sample collector 102. The annular shell 106 is configured to interface with (e.g., mate with and/or couple to) a sample tube so that contents of the collection chamber 114 (i.e., recaptured fluid and collected sample) can be emptied into the sample tube. For example, the annular shell 106 may include a coupling interface (e.g., cooperative threading 121) configured to secure the NSC apparatus 100 to an open end of the sample tube, wherein a distal end of the NSC apparatus 100 is pointed into the sample tube so that the contents of the collection chamber 114 can be transferred (e.g., poured from opening 107) into the sample tube.

In embodiments, the nasal pathway irrigator 104 and the nasal sample collector 102 are nested within the annular shell 106 and supported by an annular base 118 that includes the irrigation port 116. The annular shell 106 may include two coupling interfaces: (1) a first coupling interface (e.g., the irrigation port 116) for connecting the irrigation chamber 110 to a fluid actuator that is configured to direct fluid along the irrigation path 108 defined by the irrigation chamber 110; and (2) a second coupling interface (e.g., cooperative threading 121) for securing the NSC apparatus 100 to a sample tube with its distal end pointed into the sample tube so that contents of the collection chamber 114 can be transferred into the sample tube via the opening 107 at the distal end of the collection chamber 114.

The NSC apparatus 100 may further include a plug 124 that is configured to seal the irrigation port 116 when the irrigation chamber 110 is decoupled from the fluid actuator 202. This allows the NSC apparatus 100 and a sample tube to be fully sealed by securing the NSC apparatus 100 to the sample tube and using the plug 124 to close/seal the irrigation port 116. Otherwise, the irrigation port 116 would remain open and there would be a risk of the sample leaking out and/or a risk of contaminants leaking in. In some embodiments, the plug 124 may be tethered to the NSC apparatus 100. For example, the plug 124 may be tethered to the annular shell 106 by a tether 122 (e.g., a thin plastic strip, cord/string, etc.). In embodiments, the tether 122 is molded/fused to the annular shell 106. Alternatively, the plug 124 may be separable from the NSC apparatus 100. Other types of seals can also be used, such as a sticker, a stopper, a one-way valve, an adjustable valve, a cooperatively threaded cap, a snap-on cap, etc.

In some embodiments, the NSC apparatus 100 is formed by a single mold/print. For example, the NSC apparatus 100 may be entirely formed by a continuous 3D printed structure. Alternatively, portions of the NSC apparatus 100 may be separately printed or cast and then assembled and/or fused together. For example, the nasal sample collector 102, nasal pathway irrigator 104, or annular shell 106, annular base 118, plug 122, tether 124, or any portion of a component, may be printed or cast separately and then assembled and/or fused together to create the final device. Separately manufacturing portions of the NSC apparatus 100 may have advantages for injection molding processes because simpler molds and/or less material can be utilized.

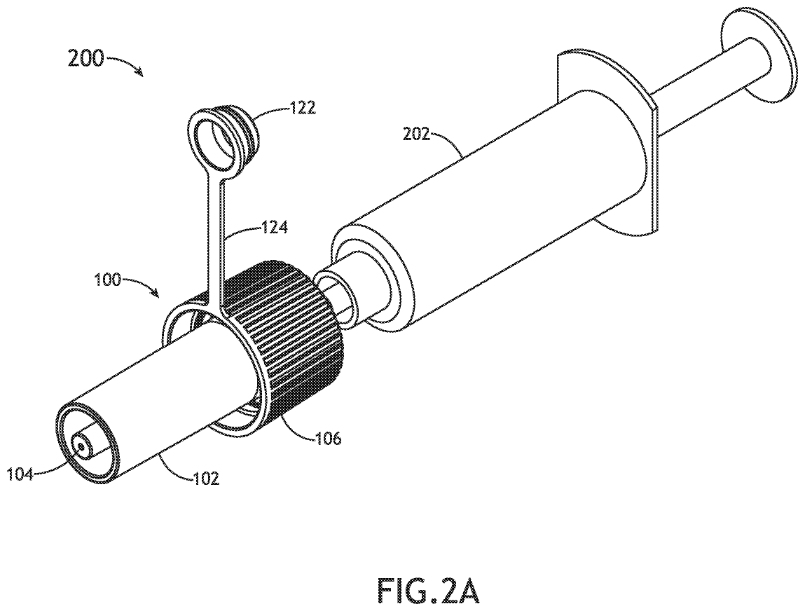

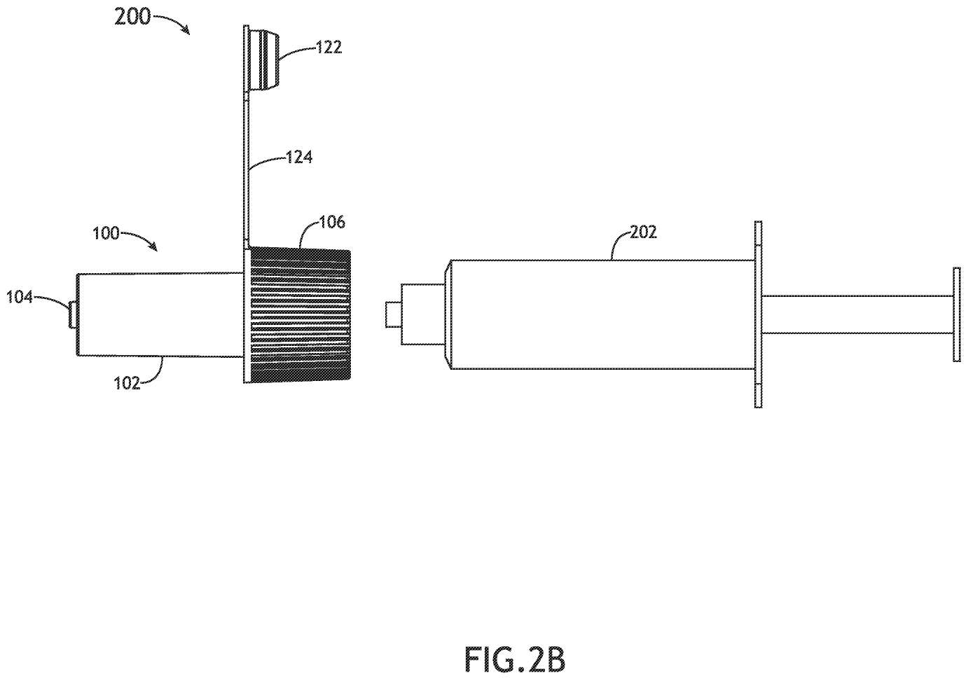

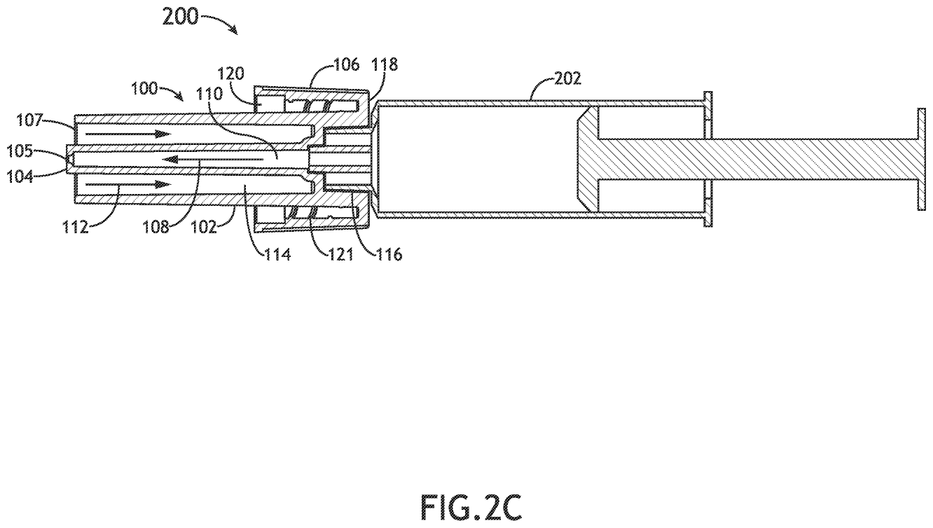

Referring now to FIGS. 2A through 2C, a system 200 (e.g., a diagnostic kit) may include the NSC apparatus 100 with a fluid actuator 202 that is configured to interface with (e.g., mate with and/or couple into) the irrigation port 116. The irrigation port 116 may be configured to interface with the fluid actuator 202 via a friction-fit connection (sometimes also referred to as an interference or tension fit connection). For example, the port diameter may be nearly the same size as the diameter of the distal tip/end of the fluid actuator 202 so that the distal tip/end of the fluid actuator 202 fits tightly within the irrigation port 116. Alternatively, or additionally, the irrigation port 116 may include a locking mechanism (e.g., a luer lock or luer slip connector).

As shown in FIG. 2C, a distal end of the fluid actuator 202 is configured to extend into the irrigation port 116. In preferred embodiments, this creates a friction-fit coupling between the fluid actuator 202 and irrigation port 116 to establish a sealed fluid connection between the fluid actuator 202 with the irrigation chamber 110. Although a friction-fit coupling is preferred, in alternative embodiments, the irrigation port 116 may include a connector, such as a luer lock or luer slip connector, or the like.

The fluid actuator 202 may be preloaded with the fluid (e.g., saline solution). This fluid can be transferred from the fluid actuator 202 to the irrigation chamber 110 to flow through the irrigation path 108. Alternatively, the fluid may be preloaded in the irrigation chamber 110, and the fluid actuator 202 may be configured to supply air or another fluid that is used to push the fluid from the irrigation chamber 110 through the irrigation path 108.

In some embodiments, the fluid actuator 202 is a syringe including a syringe barrel and a plunger configured to drive fluid through the syringe barrel. Although a syringe-type actuator is preferred, in other embodiments, the fluid actuator 202 may be a bulb-type actuator (e.g., saline bulb), a pump, or any other fluid actuator. Much like the syringe actuator embodiments described above, any other type of fluid actuator may be loaded with the irrigation fluid and/or another fluid for driving the irrigation fluid through the irrigation path 108.

When the fluid actuator 202 is coupled to the irrigation chamber 110 via the irrigation port 116, the fluid actuator 202 can be used to direct fluid along the irrigation path 108 running through the irrigation chamber 110 to deliver the fluid into the nasal pathway in order to dislodge a sample from within the nasal pathway. The nasal sample collector 102 then receives the sample by recapturing at least a portion of the fluid (now containing the sample) within the collection chamber 114 via the interface (e.g., opening 107) between the outer rim of the collection chamber 114 and the nasal pathway. The fluid is preferably directed into the nasal pathway at an incline so that at least a portion of the fluid will flow back down into the collection chamber 114.

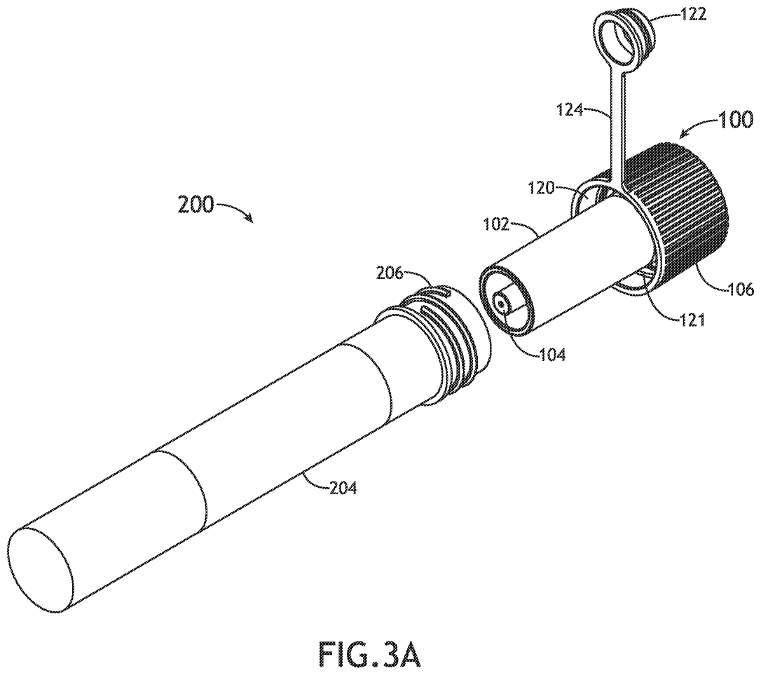

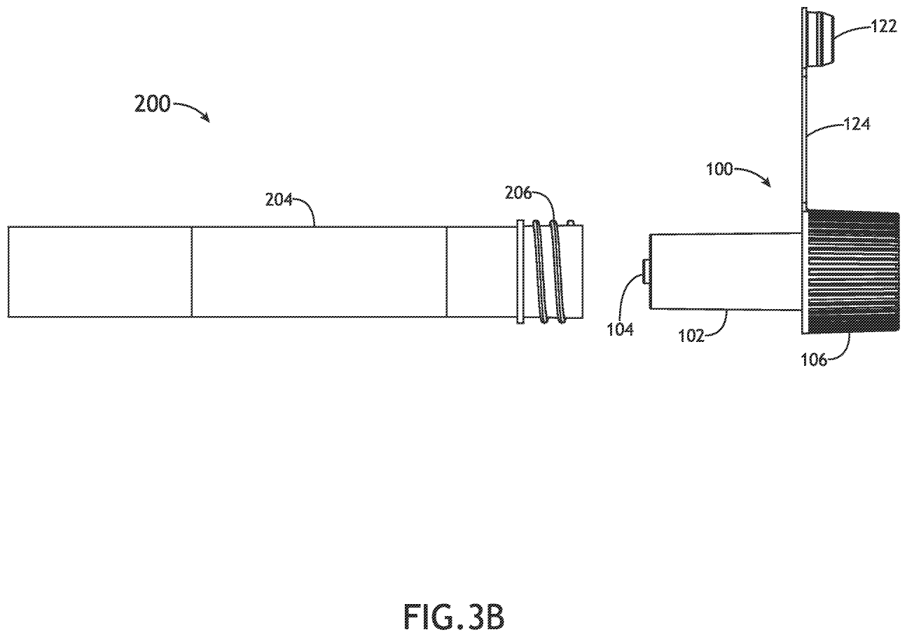

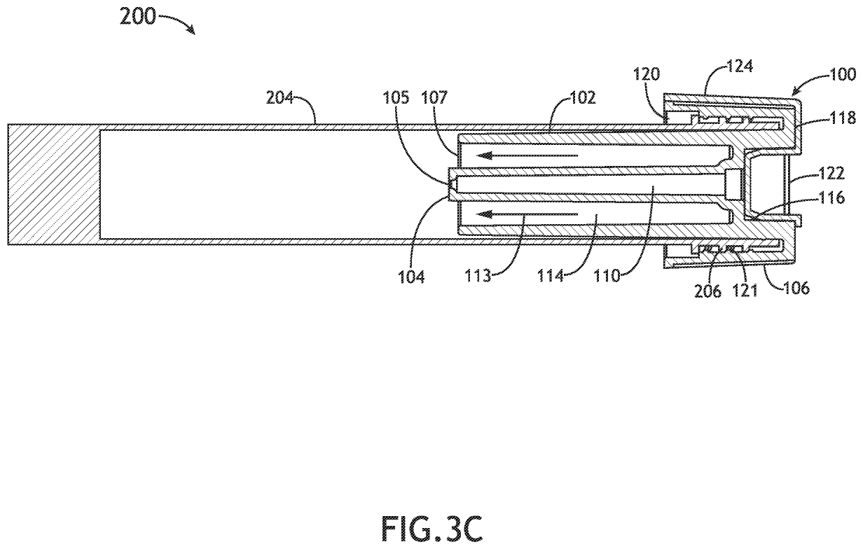

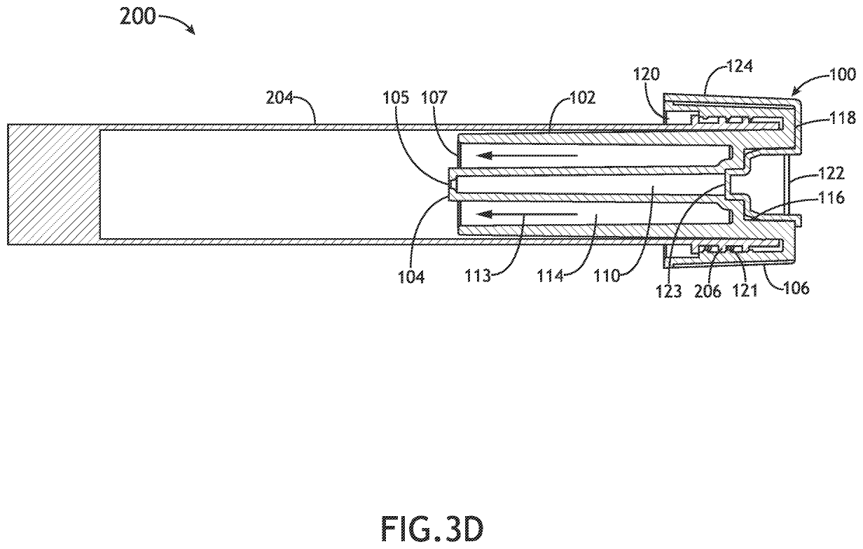

As shown in FIGS. 3A through 3C, the system 200 may further include a sample tube 204 that is configured to mate with the annular shell 106 via a coupling interface. After collecting a sample, the annular shell 106 may be interfaced with (e.g., mated with or coupled to) the sample tube 204 so that contents of the collection chamber 114, including recaptured fluid and the collected sample, can be emptied into the sample tube 202.

In embodiments, the coupling interface comprises cooperative threading 121 on an inner surface of the annular shell 106, wherein the sample tube 204 also includes cooperative threading 206 that is configured to mate with the cooperative threading 121 of the annular shell 106. The NSC apparatus 100 may be fastened (e.g., screwed on) to the sample tube 204 by screwing cooperative threading 121 into cooperative threading 206, or vice versa (see FIG. 3C). In other embodiments, the coupling interface may include an O-ring coupling interface, an interference fit coupling interface, a snap-fit coupling interface, a mechanical coupling interface (e.g., using one or more camps or other mechanical fasteners), a magnetic coupling interface, or any combination thereof.

The annular shell 106 forms a cap-like structure around the nasal sample collector 102 with an annular cavity 120 formed between the annular shell 106 and the outer body of the collection chamber 114 of the nasal sample collector 102. In embodiments, the annular shell 106 and the nasal sample collector 102 may be part of the same structure such that the annular shell 106 seamlessly extends from the outer body of the collection chamber 114 of the nasal sample collector 102. In other embodiments, the annular shell 106 and the nasal sample collector 102 may be separately produced (e.g., printed or molded) and then assembled together. The same is true of the nasal sample collector 102, the nasal pathway irrigator 104, and the annular base 118. The annular shell 106 may be connected to the nasal sample collector 102 by the annular base 118 which forms a continuous seal around the outer body of the collection chamber 114. As a result, the annular cavity 120 may be closed at one end (at the annular base 118) and open on the other end (at the distal end of the annular cavity 120).

As shown in FIG. 3C, the annular shell 106 and cavity 120 are configured to interface with the sample tube 204 so that contents of the collection chamber 114 can be transferred (along fluid path 113) from the collection chamber 114 into the sample tube 204 when the NSC apparatus 100 is mated with and/or coupled to the sample tube 204 with the distal end of the NSC apparatus 100, including opening 107, pointed into the sample tube 204. In embodiments, the sample tube 204 may be a specialized specimen collection and/or analysis tube. The sample tube 204 can also be any other type of fully/partially tubular container (e.g., test tube, jar, jug, beaker, etc.) or transient fluid transport device (e.g., flexible tube, pipe, hose, etc.).

The annular shell 106 (or another portion of the NSC apparatus 100) may be configured to be coupled to the sample tube 204 so that a portion of the sample tube 204 is secured within the annular cavity 120. For example, in the embodiment illustrated in FIG. 3C, the NSC apparatus 100 screws onto the sample tube 204 via threading 121 formed on the inner surface of the annular shell 106 and complementary threading 206 formed on an outer surface of the sample tube 204. In other embodiments, the threading may be on an outer surface of the nasal sample collector 102 and an inner surface of the sample tube 204.

The coupling interface allows the NSC apparatus 100 to seal the sample tube 204 so that the sample tube 204 can be used to safely transport fluid containing specimen collected from a nasal pathway via the NSC apparatus 100. Additionally, the plug 124 can be used to seal the irrigation port 116 when the irrigation chamber 110 is decoupled from the fluid actuator 202. This allows the NSC apparatus 100 and a sample tube to be fully sealed by securing the NSC apparatus 100 to the sample tube while also using the plug 124 to close/seal the irrigation port 116. Otherwise, the irrigation port 116 may remain open resulting in a risk of the sample leaking out and/or a risk of contaminants leaking in.

As shown in FIG. 3C, the plug 124 may comprise a substantially cylindrical section that extends into a similarly sized and shaped proximal portion of the irrigation port 116. In some embodiments, such as in FIG. 3D, the plug 124 may further include a second substantially cylindrical section 123 that extends from the first substantially cylindrical section, deeper, into a similarly sized and shaped distal portion of the irrigation port 116, wherein the distal portion of the irrigation port 116 has a smaller diameter than the proximal portion of the irrigation port 116. In this regard, the plug 124 may have a similar size and shape to a distal end/tip of the fluid actuator 202.

Referring again to FIG. 1C, the NSC apparatus 100 may include one or more annular bead seals 132 (or gaskets) inside the annular cavity 120 (e.g., on an outer surface of the nasal sample collector 102 and/or an inner surface of the annular shell 120), on the plug 124, and/or on an inner surface of the irrigation port 116 to further seal the NSC apparatus 100 and prevent leakage during sample collection, transfer to the sample tube 204, and/or transport to a specimen storage/testing facility.

The NSC apparatus 100 allows for a sample to be collected, secured within the sample tube 204, and transported with little to no risk of contamination or potentially hazardous exposure to the handler. In some embodiments, the sample tube 204 or collection chamber 114 may also contain a specimen preservation agent and/or a testing agent so that the sample collected from the nasal pathway is preserved or immediately tested within the sample tube 204 or collection chamber 114. For example, the sample tube 204 or collection chamber 114 may be prefilled with viral transport media (VTM) and/or an antigen detection agent.

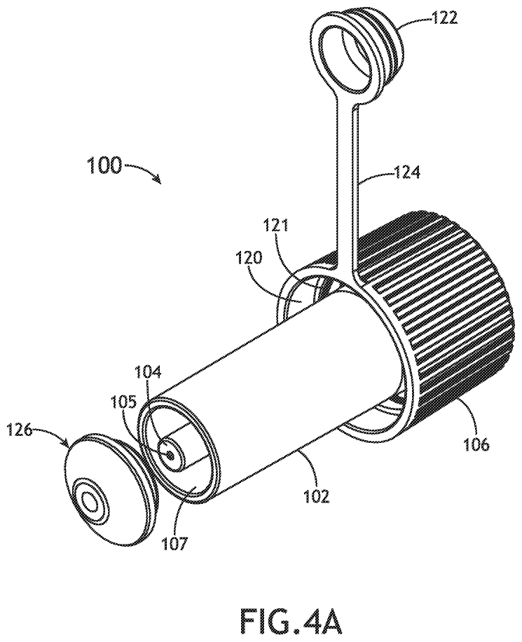

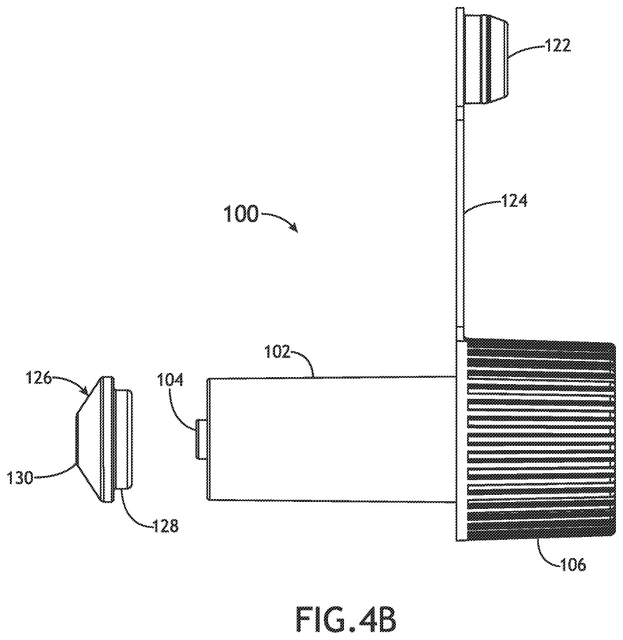

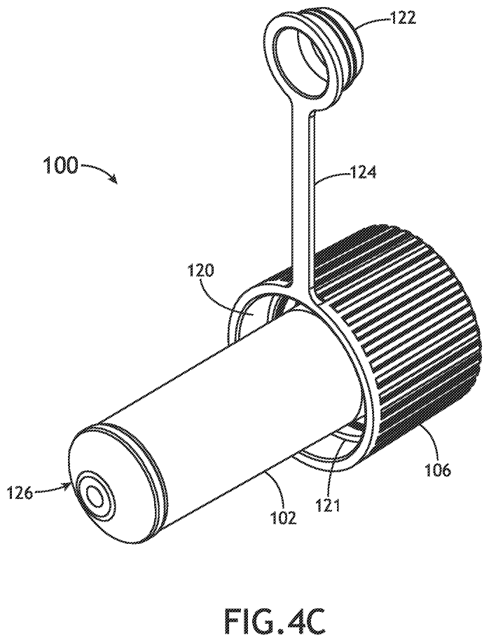

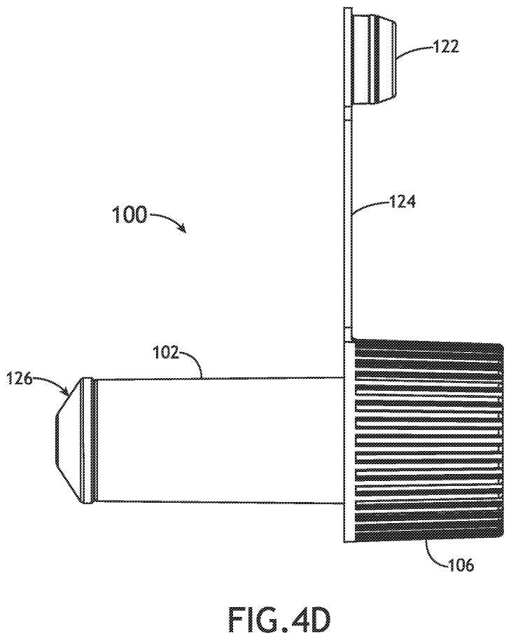

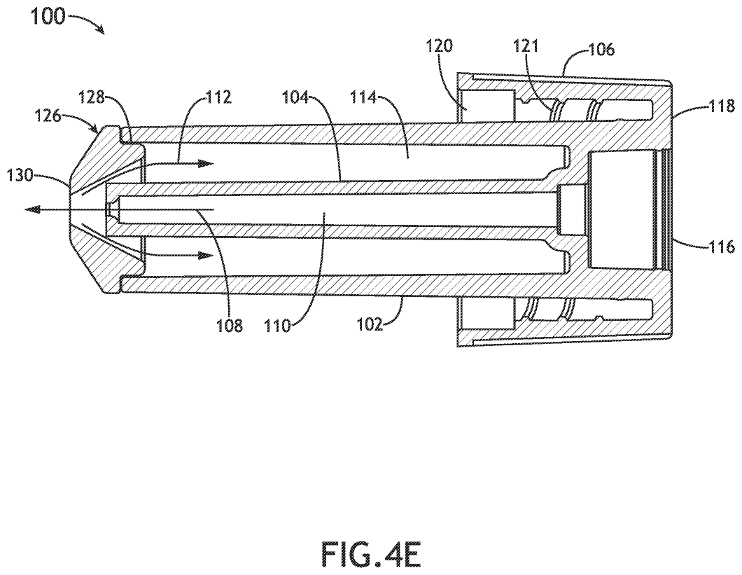

As shown in FIGS. 4A through 4E, in some embodiments, the NSC apparatus 100 may be further equipped with one or more tip adapters 126 that can be coupled to the distal end of the NSC apparatus 100 for pediatric applications or more generally to accommodate differently sized/shaped nostrils. In embodiments, a tip adapter 126 is configured to be coupled to the outer rim of the nasal sample collector 102, wherein the tip adapter 126 has distal opening/nozzle 130 with a smaller diameter than the outer rim of the nasal sample collector 102. The tip adapter 126 may also include an annular base 128 that is configured to fit tightly within the opening 107 defined by the outer rim of the nasal sample collector 102. For example, the annular base 128 of the tip adapter 126 may have an outer diameter that is substantially equal to the inner diameter of the opening 107 so that the tip adapter 126 can be attached to the nasal sample collector 102 via a friction-fit coupling. This structural configuration allows the tip adapter 126 to be plugged into the opening 107. As shown in FIG. 4E, the tip adapter 126 modifies the collection path 112 that leads to the collection chamber 114 in a manner that accommodates smaller nostrils.

A method of nasal specimen collection may employ the NSC apparatus 100 or a similarly structured device. The method may include the steps of: (1) providing a NSC apparatus 100 that includes a nasal pathway irrigator 104 and a nasal sample collector 102 nested within an annular shell 106 that has a coupling interface (e.g., cooperative threading 121); (2) inserting a distal end of the nasal sample collector 102 into a nasal pathway, wherein the nasal sample collector 102 includes a collection chamber 114 that surrounds the nasal pathway irrigator 104 and has an outer rim that is configured to interface with the nasal pathway; (3) directing fluid into the nasal pathway via the nasal pathway irrigator 104 in order to dislodge a sample from within the nasal pathway (e.g., by pushing fluid through along an irrigation path 108 defined by an irrigation chamber 110 of the nasal pathway irrigator 104 with a fluid actuator 202 that is coupled to an irrigation port 116 at a proximal end of the NSC apparatus 100); (4) recapturing at least a portion of the fluid within the collection chamber 114 of the nasal sample collector 102 in order to collect the sample that was dislodged from within the nasal pathway (e.g., via an opening 107 defined by the outer rim of the collection chamber 114); and (5) coupling the annular shell 106 to a sample tube 204 via the coupling interface (e.g., cooperative threading 121 and 206) to transfer contents of the collection chamber 114, including recaptured fluid containing the sample, to the sample tube 204 and to seal the contents within the sample tube 204. Before or after step (5), the plug 124 may also be used to seal the irrigation port 116.

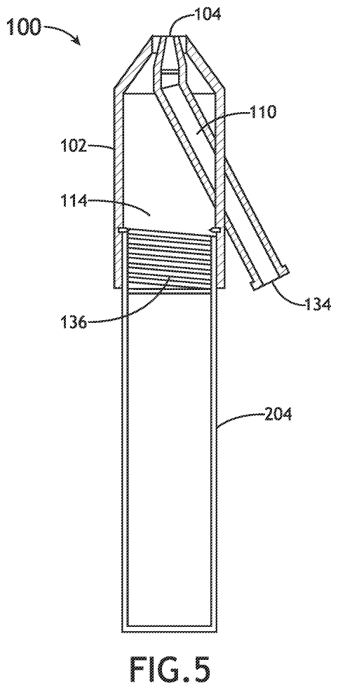

FIG. 5 illustrates an alternative embodiment of the NSC apparatus 100, where the collection chamber 114 defines a fluid path that flows directly into a sample tube 204 that is coupled to the NSC apparatus 100 via a coupling interface (e.g., cooperative threading 136) a proximal end of the collection chamber 114. A syringe 202 may be used to direct fluid through the irrigation chamber 110 via an irrigation port 134 extending from an outer sidewall of NSC apparatus 100. The irrigation chamber 110 defines a fluid path that extends into the collection chamber 114 from the irrigation port 134 at an angle; while the collection chamber 114 defines a fluid path that flows from distal end of the NSC apparatus 100 straight through the collection chamber 114 and into the sample tube 204 that is coupled thereto. In this embodiment of the NSC apparatus 100, contents of the collection chamber 114 are transferred immediately into the sample tube 204. After collecting a sample, the distal end of the NSC apparatus 100 may be sealed (e.g., using a plug/cap structure) so that the NSC apparatus 100 seals the sample tube 204. The irrigation port 134 can also be sealed (e.g., using a plug/cap structure). The sample tube 204 is then ready for storage or transport to a sample storage/testing facility.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

1. A nasal specimen collection apparatus, comprising:
a nasal pathway irrigator including an irrigation chamber that defines an irrigation path for directing fluid into a nasal pathway in order to dislodge a sample from within the nasal pathway;
a nasal sample collector including a collection chamber that surrounds the nasal pathway irrigator, wherein the collection chamber has an outer rim that is configured to interface with the nasal pathway, and wherein the collection chamber defines a collection path for recapturing at least a portion of the fluid in order to collect the sample that was dislodged from within the nasal pathway; and
an annular shell that surrounds the nasal sample collector with an annular cavity formed in between the annular shell and the collection chamber of the nasal sample collector, wherein the annular shell is configured to interface with a sample tube so that contents of the collection chamber can be emptied into the sample tube.

2. The nasal specimen collection apparatus of claim 1, wherein the nasal pathway irrigator and the nasal sample collector are nested within the annular shell and supported by an annular base that includes an irrigation port for coupling a fluid actuator to the irrigation chamber of the nasal pathway irrigator.

3. The nasal specimen collection apparatus of claim 2, further comprising:
a plug tethered to the annular shell, wherein the plug is configured to seal the irrigation port when the irrigation chamber is decoupled from the fluid actuator.

4. The nasal specimen collection apparatus of claim 3, further comprising one or more bead seals on an inner surface of the irrigation port and/or an outer surface of the plug.

5. The nasal specimen collection apparatus of claim 1, wherein the irrigation chamber and the collection chamber are coaxial.

6. The nasal specimen collection apparatus of claim 1, wherein the nasal pathway irrigator extends past the outer rim of the nasal sample collector to prevent recaptured fluid from backflowing from the collection chamber into the irrigation chamber.

7. The nasal specimen collection apparatus of claim 1, wherein an outer body of the collection chamber is tapered in the range of 0.5 to 5 degrees from a proximal end of the outer body to a distal end of the outer body.

8. The nasal specimen collection apparatus of claim 1, wherein an interior of the irrigation chamber includes a first section that is tapered in the range of 0.1 to 5 degrees over a distance in the range of 20 to 50 millimeters and a second section that is tapered in the range of 15 to 45 degrees over a distance in the range of 0.5 to 5 millimeters.

9. The nasal specimen collection apparatus of claim 1, wherein the annular shell includes a coupling interface for securing the annular shell to the sample tube.

10. The nasal specimen collection apparatus of claim 9, wherein the coupling interface comprises cooperative threading on an inner surface of the annular shell.

11. The nasal specimen collection apparatus of claim 10, wherein the coupling interface further comprises one or more bead seals between the nasal sample collector and the outer annular shell.

12. The nasal specimen collection apparatus of claim 1, further comprising:
a tip adapter configured to be coupled to the outer rim of the nasal sample collector, wherein the tip adapter has a distal opening with a smaller diameter than the outer rim of the nasal sample collector.

13. A nasal specimen collection system, comprising:
a fluid actuator;
a sample tube; and
a nasal specimen collection apparatus, the nasal specimen collection apparatus comprising:
a nasal pathway irrigator including an irrigation chamber that defines an irrigation path for directing fluid into a nasal pathway in order to dislodge a sample from within the nasal pathway;
a nasal sample collector including a collection chamber that surrounds the nasal pathway irrigator, wherein the collection chamber has an outer rim that is configured to interface with the nasal pathway, and wherein the collection chamber defines a collection path for recapturing at least a portion of the fluid in order to collect the sample that was dislodged from within the nasal pathway; and
an annular shell that surrounds the nasal sample collector with an annular cavity formed in between the annular shell and the collection chamber of the nasal sample collector, wherein the nasal pathway irrigator and the nasal sample collector are nested within the annular shell and supported by an annular base that includes an irrigation port for coupling the fluid actuator to the irrigation chamber of the nasal pathway irrigator, and wherein the annular shell is configured to interface with the sample tube so that contents of the collection chamber can be emptied into the sample tube.

14. The nasal specimen collection system of claim 13, wherein the fluid actuator comprises a syringe configured to mate with the irrigation port of the nasal specimen collection apparatus.

15. The nasal specimen collection system of claim 13, wherein the nasal specimen collection apparatus further comprises:
a plug tethered to the annular shell, wherein the plug is configured to seal the irrigation port when the irrigation chamber is decoupled from the fluid actuator.

16. The nasal specimen collection system of claim 13, wherein the annular shell includes a coupling interface for securing the annular shell to the sample tube.

17. The nasal specimen collection system of claim 16, wherein the coupling interface comprises cooperative threading on an inner surface of the annular shell.

18. The nasal specimen collection system of claim 17, wherein an open end of the sample tube is cooperatively threaded and configured to mate with the cooperative threading on the inner surface of the annular shell.

19. The nasal specimen collection system of claim 13, wherein the nasal specimen collection apparatus further comprises:
a tip adapter configured to be coupled to the outer rim of the nasal sample collector, wherein the tip adapter has a distal opening with a smaller diameter than the outer rim of the nasal sample collector.

20. A method of nasal specimen collection, comprising the steps of:
inserting a distal end of a nasal specimen collection apparatus into a nasal pathway, wherein the nasal specimen collection apparatus includes a nasal pathway irrigator and a nasal sample collector nested within an annular shell that has a coupling interface, and wherein the nasal sample collector includes a collection chamber that surrounds the nasal pathway irrigator and has an outer rim that is configured to interface with the nasal pathway;
directing fluid into the nasal pathway via the nasal pathway irrigator in order to dislodge a sample from within the nasal pathway;
recapturing at least a portion of the fluid within the collection chamber of the nasal sample collector in order to collect the sample that was dislodged from within the nasal pathway; and
coupling the annular shell to a sample tube via the coupling interface to transfer contents of the collection chamber to the sample tube and to seal the contents within the sample tube.
